(19) United States
(12) Patent Application Publication
(10) Pub. No.: US 2009/0269780 A1
(43) Pub. Date: Oct. 29, 2009

(54) Method for Creating a Standard for Multiple Analytes Found in a Starting Material of Biological Origin

(75) Inventors: Keld Sorensen, Austin, TX (US); Patricia Hebert, Cedar Park, TX (US)

Correspondence Address: FULBRIGHT & JAWORSKI L.L.P., 600 CONGRESS AVE., SUITE 2400, AUSTIN, TX, 78701, US

(73) Assignee: Luminex Corporation, Austin, TX (US)

(21) Appl. No.: 12/428,230

(22) Filed: Apr. 22, 2009

Related U.S. Application Data
(60) Provisional application No. 61/047,232, filed on Apr. 23, 2008.

(51) Int. Cl. G01N 33/53; G01N 31/00

(52) U.S. Cl. 435/7.1; 436/17; 436/15

(57) ABSTRACT

The invention provides a method for creating a standard for multiple analytes comprising treating a portion of a sample to substantially remove analytes of interest to produce a series of specifically deficient samples; and determining and mixing an appropriate amount of the series of specifically deficient samples to create a standard. The analyte may be any substance to be measured.

The present application claims benefit of priority to U.S. Provisional Application Ser. No. 61/047,232, filed Apr. 23, 2008, the entire contents of which are hereby incorporated by reference.

### BACKGROUND OF THE INVENTION

A. Field of the Invention

The present invention relates generally to the field of molecular biology. More particularly, it concerns methods of creating a standard useful for multiplex assays. In specific embodiments, the invention concerns a method of creating a substantially uniform standard from multiple starting materials.

B. Description of Related Art

The recent appearance of multiplexed assays has created a unique problem when manufacturing standards for those assays.

In a single assay (i.e. in a singlex assay), where the analysis is of one analyte, a set of standards with known concentrations of the analyte are used to create the “standard curve” against which unknown samples are measured. Two methods are generally used for creating the standards: a) either a matrix devoid of the target analyte is found or created, into which purified analyte (standard) is spiked, or b) a sample with a known high level of analyte is used as the “high” standard, and lower levels are created by diluting this high standard into a matrix that is either devoid or low in the target analyte.

In multiplex assays, approach a) of the above listing is often used when the analyte is readily available in a pure form that allows “spiking.” This is entirely adequate when the respective target analytes can be obtained in pure form and can be handled in the spiking process. The process is simply repeated for each analyte in the multiplex profile, so that if analytes x, y and z are needed, each is spiked into the same matrix to ensure that a single standard has all the analytes needed at the appropriate levels for the assay.

A particularly complex problem is encountered when analytes can only be obtained biologically, and/or can not be readily purified or otherwise handled. In such cases, one typically tries to identify a large number of different samples that have various levels of analytes of interest, followed by a process of mixing the material so as to reach given target values for all analytes. This means that if a high standard for one analyte is picked, it may or may not have reasonable levels of the other analytes that make up the multiplexed assay, resulting in standards that can not readily be repeated and/or that do not have the desired target values. There is therefore a need for a method of consistently creating a substantially uniform standard for use with multiplex assays.

### SUMMARY OF THE INVENTION

In some aspects, the invention provides a method for creating a multiplex standard for a plurality of analytes from a starting sample comprising obtaining a sample comprising a plurality of analytes of interest; determining a concentration for each analyte of interest; creating one or more specifically deficient samples; determining the amount, if any, of the starting sample and of each specifically deficient sample required to create a multiplex standard having a substantially uniform concentration of each analyte; and mixing the amount of the starting sample and the specifically deficient samples to create the multiplex standard.

The starting sample may be any composition containing an analyte of interest. A starting sample may contain a substantially uniform or highly varied concentration of the various analytes of interest present. In certain aspects of the invention, the sample may be a bodily fluid (including but not limited to whole blood, serum, saliva, urine, sperm). In some particular embodiments, the starting sample is a blood sample. The analyte may be any substance to be measured. In particular embodiments, the analyte is a protein, an antibody, or a protease.

In some embodiments, creating the one or more specifically deficient samples may comprise treating a portion of the starting sample or a specifically deficient sample to substantially remove at least a first analyte of interest. An analyte of interest may be removed to create a biological sample devoid or at least of low concentration of that particular analyte. This is generally done with minimal effect on the analyte levels of the other analytes, however, this process does not need to be 100% specific. In certain embodiments, there are non-specific reductions in another analyte of less than 5%, 10%, 15%, 20%, 25%, 30%, 35%, or 40%. The analyte of interest need not be completely removed. An analyte is “substantially removed” when at least about 50%, 60%, 70%, 75%, 80%, 85%, 90%, 95%, 96%, 97%, 98%, 99% or 100% of the analyte is removed. The sample may be treated in any manner that would substantially remove a target analyte. In particular embodiments, the treatment comprises neutralizing the analyte, physically removing the analyte, destroying the analyte or sequestering the analyte.

In some embodiments, the analyte may be an antibody. As used herein, the term “antibody” is intended to refer broadly to any immunologic binding agent, such as IgG, IgM, IgA, IgD and IgE. In some embodiments, the antibody may be, for example, an antibody to Haemophilus influenza type b (Hib) polysaccharide and the toxoids of Clostridium tetani (Tet) and Corynebacterium diphtheriae (Dip), Streptococcus pneumonoiae, Meningoccus, Polio, Diptheria, Tetanus, HIV, HBV, HCV. One of skill in the art would be aware that various methods exist to remove the antibody from a starting sample or a specifically deficient sample. In particular embodiments, the antibody is removed from a sample or standard by neutralizing the antibody, for example by providing an antigen specific to the antibody of interest to the sample. In other embodiments, the antibody is removed from a sample or standard by physically removing the antibody, for example by immobilizing the antibody on a column having an antigen specific for the antibody. In another embodiment, the antibody is removed from a sample or standard by destroying the antibody, for example by treating the sample with a destructive agent or technique, such as a protease treatment or heat treatment. In still further embodiments, the antibody is removed from a sample or standard by sequestering the antibody, for example by incorporation of the antibody into a liposome.

In some embodiments, the analyte may be a protein. In some embodiments, the protein may be, for example, insulin, TSH, tetanus toxin or toxoid, diphtheria toxin or toxoid, pituitary hormones, trypsin or trypsinogen. One of skill in the art would be aware that various methods exist to remove the protein from the starting sample or the specifically deficient sample. In particular embodiments, the protein is removed from a sample or standard by neutralizing the protein, for example by providing a target specific to the protein of interest to the sample. In other embodiments, the protein is removed from a sample or standard by physically removing the protein, for example by immobilizing the protein on a column having a target that binds the protein. In another embodiment, the protein is removed from a sample or standard by destroying the protein, for example by treating the sample with a destructive agent or technique, such as a protease treatment or heat treatment. In still further embodiments, the protein is removed from a sample or standard by sequestering the protein, for example by incorporation of the protein into liposomes or adsorption of the protein onto a solid surface, such as silica, molecular sieves or similar.

In some embodiments, the analyte may be a protease. As used herein, the term “protease” is intended to refer broadly to any enzyme that conducts proteolysis. In some embodiments, the protease may be trypsion or chymotrypsin. One of skill in the art would be aware that various methods exist to remove or inactivate the protease from a starting sample or a specifically deficient sample. In particular embodiments, the protease is removed from a sample or standard by neutralizing the protease, for example by providing an inhibitor specific to the protease of interest to the sample. In other embodiments, the protease is removed from a sample or standard by physically removing the protease, for example by immobilizing the protease on a column having an inhibitor that binds the protease. In another embodiment, the protease is removed from a sample or standard by destroying the protease, for example by proteolysis by another protease that can more readily be inactivated or destroyed. In still further embodiments, the protease is removed from a sample or standard by sequestering the protease, for example by sequestration of the protease into a liposome.

In particular embodiments, the invention provides a method for creating a standard for multiple analytes comprising obtaining a sample comprising a plurality of analytes of interest; determining a concentration for each analyte of interest; treating a portion of the sample to substantially remove a first analyte to create a first specifically deficient sample; treating a portion of the first specifically deficient sample to substantially remove a second analyte to create a second specifically deficient sample; repeating the process to remove subsequent analytes from subsequent specifically deficient samples, thereby producing a series of specifically deficient samples; determining an amount of the starting sample and the series of specifically deficient samples required to create a standard having a desired concentration of each of the analytes; and mixing the amount of the series of specifically deficient samples to create the standard.

In some embodiments, the starting sample is treated to remove a first analyte of interest to create a first specifically deficient sample. The first, or preceding, specifically deficient sample may then be treated to remove a second analyte of interest and create a second, or successive, specifically deficient sample, or a specifically deficient sample. This process may be repeated, with each preceding specifically deficient sample being treated to remove an analyte of interest to create a successive specifically deficient sample.

For example, in some embodiments, the invention provides a method for creating a multiplex standard for three analytes of interest from a starting sample comprising obtaining a sample comprising three analytes of interest; determining a concentration for each analyte of interest; treating a portion of the starting sample to remove a first analyte of interest to create a first specifically deficient sample; treating a portion of the first specifically deficient sample to remove a second analyte of interest to create a second specifically deficient sample; determining the amount of the starting sample and each specifically deficient sample required to create a multiplex standard having a substantially uniform concentration of each analyte; and mixing the amount of the starting sample and the specifically deficient samples to create the multiplex standard.

In other embodiments, the starting sample comprises five analytes of interest. In such embodiments, the method comprises obtaining a sample comprising five analytes of interest; determining a concentration for each analyte of interest; treating a portion of the starting sample to remove a first analyte of interest to create a first specifically deficient sample; treating a portion of the first specifically deficient sample to remove a second analyte of interest to create a second specifically deficient sample; treating a portion of the second specifically deficient sample to remove a third analyte of interest to create a third specifically deficient sample; treating a portion of the third specifically deficient sample to remove a fourth analyte of interest to create a fourth specifically deficient sample; determining the amount of the starting sample and each specifically deficient sample required to create a multiplex standard having a substantially uniform concentration of each analyte; and mixing the amount of the starting sample and the specifically deficient samples to create the multiplex standard.

The analytes of interest may be removed from the standards in any order. In some embodiments, the analyte removed is any analyte in the starting sample. In other embodiments, the first analyte of interest is the analyte with the highest concentration in the starting sample, the second analyte of interest is the analyte with the highest concentration in the first specifically deficient sample, the third analyte of interest is the analyte with the highest concentration in the second specifically deficient sample, and the fourth analyte of interest is the analyte with the highest concentration in the third specifically deficient sample.

The method of the invention provides for determining the amount of the starting sample and each specifically deficient sample required to create a multiplex standard having a substantially uniform concentration of each analyte. This may be achieved by any method known to those of skill in the art. For example, the amount of the starting sample and each specifically deficient sample required to create a multiplex standard having a substantially uniform concentration of each analyte may be determined by selecting a desired concentration for each analyte, and determining the amounts of the starting sample and each specifically deficient sample required based on that desired concentration, or target value. The ratio of the actual value of the analyte to the target value of the analyte (“actual to target value ratio”) of each analyte can be averaged to give an average ratio for the mixed multiplex standard, which is an indication of the uniformity of the standard. For example, a completely uniform multiplex standard where each analyte has the exact same concentration as the desired concentration, will have an average actual to target value ratio of 1.0 with a range of actual to target value ratios of 1.0 to 1.0 A less uniform multiplex standard may have an average actual to target value ratio of 0.74, where the range of actual to target value ratios is from 0.30 to 1.90. In particular embodiments of the concentration, a substantially uniform multiplex standard may have an actual to target value ratio in the range of 0.50 to 1.50. In further embodiments, the substantially uniform multiplex standard may have an actual to target ratio in the range of 0.75 to 1.25.

The desired concentration should be determined based on a concentration that is biologically relevant for the analyte to be investigated or measured and the assay to be performed. For example, in some illustrative embodiments, the desired concentration of each analyte in the multiplex standard may be 25,000 μg/ml. In such embodiments, in the multiplex standard, each analyte may, for example, have a concentration between 1,000 to in excess of 50,000 μg/ml. This would provide an average actual to target ratio of 0.04 to over 2.0. In a multiplex standard having a substantially uniform concentration of each analyte, the concentration of each analyte may be between 20,000 to 30,000 μg/ml. This would provide an average actual to target ratio of 0.80 to 1.20. In other embodiments, the desired concentration of each analyte in the multiplex standard may be 5,000 μg/ml. In such embodiments, in the multiplex standard, each analyte may have a concentration between 1,000 to 50,000 μg/ml. This would provide an average actual to target ratio of 0.20 to over 10. In a multiplex standard having a substantially uniform concentration of each analyte, the concentration of each analyte may be between 3,000 to 7,000 μg/ml. This would provide an average actual to target ratio of 0.60 to 1.40.

In particular embodiments, the methods of the invention further comprising identifying the analytes of interest that have outlier concentrations. An outlier is a statistical observation that is markedly different in value or is numerically distant from the rest of the values in a given sample. Illustratively, an outlier concentration may be determined in comparison to a desired concentration. For example, it may be desirable to identify samples having a concentration that is significantly higher or lower than the desired concentration as an outlier concentration. In an illustrative embodiment, an outlier has a concentration that is significantly higher or lower than the desired concentration and may have an actual to target value ratio of less than 0.50 or greater than 1.50. In other embodiments, the outlier may have a concentration that may have an actual to target value ratio of less than 0.75 or greater than 1.25. In other embodiments, the outlier may be identified in comparison to any standard known to those of skill in the art.

In such embodiments, the starting sample or samples may be treated to remove only the analytes having an outlier concentration. For example, the method may comprise obtaining a sample comprising a plurality of analytes of interest; determining a concentration for each analyte of interest; identifying the analytes of interest that have outlier concentrations; creating one or more specifically deficient samples by treating a starting sample or a specifically deficient sample to remove at least one analyte identified as having an outlier concentration; determining the amount of the starting sample and each specifically deficient sample required to create a multiplex standard having a substantially uniform concentration of each analyte; and mixing the amount of the starting sample and the specifically deficient samples to create the multiplex standard. Alternatively, the starting sample or a specifically deficient sample may be treated to remove the analytes having an outlier concentration first, and then the starting sample or a specifically deficient sample may be treated to remove any additional analytes desired.

In some embodiments, creating the one or more specifically deficient samples comprises creating a series of N−1 specifically deficient samples in which each successive, specifically deficient sample has had substantially removed the analyte of interest having the highest concentration in the preceding starting sample or the preceding specifically deficient sample.

In some embodiments, the invention further comprises making a series of dilutions of the multiplex standard. This series of dilutions may be used to create a standard curve for the analysis of unknown samples. A standard curve is generated by plotting assay data that relates a known concentration of an analyte to an empirical value and is used to determine the concentration of a substance, particularly proteins and DNA.

In still further embodiments, the invention provides a method for creating a multiplex standard for a plurality of analytes from a plurality of starting samples comprising obtaining a plurality of starting samples comprising a plurality of analytes of interest; determining a concentration for each analyte of interest in each starting sample; creating one or more specifically deficient samples; determining the amount of each specifically deficient sample required to create a multiplex standard having a desired concentration of each analyte; and mixing the amount of the starting sample and the specifically deficient samples to create the multiplex standard. In some embodiments, creating the one or more specifically deficient samples comprises treating a portion of at least one of the starting samples to substantially remove at least one of the analytes of interest.

The invention further provides for the creation of a multiplex standard utilizing a plurality of starting samples. In some embodiments, the invention provides for 1, 2, 3, 4, 5, 6, 7, 8, 9, 10, 11, 12, 13, 14, 15, 16, 17, 18, 19, or 20 or more starting samples or any range derivable therein. In a particular embodiment, the multiplex standard is created from 5 starting samples. In such an embodiment, creating the one or more specifically deficient samples may comprise treating a portion of the first, second, third, fourth, and/or fifth starting samples to remove at least one analyte of interest from the starting sample to create a first, second, third, fourth, and/or fifth specifically deficient sample. In another embodiment, the multiplex standard is created from 10 starting samples, wherein creating the one or more specifically deficient samples comprises treating a portion of the first, second, third, fourth, fifth, sixth, seventh, eighth, ninth and/or tenth starting samples to remove at least one analyte of interest from the starting sample to create a first, second, third, fourth, fifth, sixth, seventh, eighth, ninth and/or tenth specifically deficient sample.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. Results using PS-1 Affinity Column. Signal for antibody to PS-1 was reduced to <30% original signal.

FIG. 2. Results using PS-4 Affinity Column. Signal for antibody to PS-4 was reduced to <20% original signal.

FIG. 3. Results using PS-9 Affinity Column. Signal for antibody to PS-9 was reduced to <15% original signal.

FIG. 4. Results using PS-12 Affinity Column. PS-12 affinity column non-specifically reduced all PS antibodies.

FIG. 5. Results using PS-23 Affinity Column. PS-23 affinity column non-specifically reduced all PS antibodies.

FIG. 6. Results using PS-57 Affinity Column. Signal for antibody to PS-57 was reduced to <10% original signal.

FIG. 7. Percentage of Control Detected Antibody Levels. Samples were incubated with corresponding polysaccharide.

### DESCRIPTION OF ILLUSTRATIVE EMBODIMENTS

### I. The Present Invention

The invention provides an approach to preparing material for use in establishing a standard curve for biological assays where the target analytes can not be created synthetically or where these target analytes can not readily be purified and/or manipulated in a production setting. One example is a serology assay, where antibody levels against various analytes have to be produced in a living organism, such as a human being, but where the level of each antibody so produced can not be controlled or regulated. The invention utilizes removal of the target analyte(s) to produce specifically deficient solutions which can then be used to prepare mixtures containing specific and substantially uniform levels of target analytes.

In certain embodiments, the present invention provides a method for creating standards for multiplex assays. As used herein, the phrase “multiplex” or grammatical equivalents refers to the parallel detection, analysis or amplification of more than one target analyte of interest per sample. Analysis of multiple different analytes (multiplex) may be performed simultaneously. Detection is performed on a variety of platforms, including but not limited to microarrays and bead arrays.

### II. Producing the Specifically Deficient Samples

In some aspects, the current invention provides for creating one or more specifically deficient samples comprising treating a portion of a starting sample or a specifically deficient sample to substantially remove an analyte of interest. The analyte may be any substance to be measured, for example a protein, an antibody or a protease. Methods of removing a protein, an antibody, or a protease from a sample are well known to those of skill in the art, and include, but are not limited to, those methods described below. Prior to removing a specific analyte to create a specifically deficient sample, the concentration of those analytes of interest may be known/quantified.

A. Quantification of the Analytes of Interest

The methods of the present invention provide for determining a concentration for each analyte of interest. One of skill in the art would understand that various methods exist for quantifying any analyte of interest, including but not limited to those described below.

1. ELISA

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle and Ben-Zeev, 1999; Gulbis and Galand, 1993; De Jager et al., 1993; and Nakamura et al., 1987, each incorporated herein by reference.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241, each incorporated herein by reference. Of course, one may find additional advantages through the use of a secondary binding ligand such as a second antibody and/or a biotin/avidin ligand binding arrangement, as is known in the art.

The selective antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the antibody is used to form secondary immune complexes, as described above. After washing, the secondary immune complexes may be contacted with a third binding ligand or antibody that has binding affinity for the second antibody, again under effective conditions and for a period of time sufficient to allow the formation of immune complexes (tertiary immune complexes). The third ligand or antibody is typically linked to a detectable label, allowing detection of the tertiary immune complexes thus formed. This system may provide for signal amplification if this is desired.

As detailed above, immunoassays, in their most simple and/or direct sense, are antibody binding assays. Certain preferred immunoassays are the various types of enzyme linked immunosorbent assays (ELISAs) and/or radioimmunoassays (RIA) known in the art.

In one exemplary ELISA, the selective antibodies of the invention are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA”. Detection may also be achieved by the addition of a second selective antibody, followed by the addition of a third antibody that has binding affinity for the second antibody, with the third antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer and relating the value to a similar value produced using a known amount of analyte.

2. Mass Spectrometry

Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). Mass spectrometry (MS), because of its extreme selectivity and sensitivity, has become a powerful tool for the quantification of a broad range of bioanalytes including pharmaceuticals, metabolites, peptides and proteins.

Quantification by mass spectrometry is achieved by the use of specifically designed techniques known to those of skill in the art. One example is the use of spike-in controls. A known quantity of an isotopically labeled form of the desired analyte is added to a solution to create a control, which can then be used to correlate peak height with molecular concentrations. The mass spectrometer may be set to monitor only the mass and charge of the analytes of interest, known as selected ion monitoring (SIM).

3. Western Blot Analysis

Western blot analysis is an established technique that is commonly employed for analyzing and identifying proteins. The proteins are first separated by electrophoresis in polyacrylamide gel, then transferred (“blotted”) onto a nitrocellulose membrane or treated paper, where they bind in the same pattern as they formed in the gel. The antigen is overlaid first with antibody, then with anti-immunoglobulin or protein A labeled with a radioisotope, fluorescent dye, or enzyme. One of ordinary skill in the art would be familiar with this commonly used technique for quantifying protein in a sample.

B. Altering Levels of the Analyte of Interest Methods of removing an analyte from a starting sample or a specifically deficient sample are well known to those of skill in the art and include, but are not limited to, those methods described below.

1. Neutralizing the Analyte of Interest

In particular embodiments, the analyte is removed from a sample or standard by neutralizing the antibody. The analyte may be neutralized or inactivated by any method known to those of skill in the art, including but not limited to introducing into the sample a molecule that binds the analyte.

2. Physically Removing the Analyte of Interest

In other embodiments, the analyte is removed from a sample or standard by physically removing the analyte of interest. The analyte may be physically removed by any method known to those of skill in the art, including but not limited to those methods described below.

In some embodiments, the analyte of interest may be removed from a sample or standard by immobilizing the analyte on a solid support having a target that binds the analyte. The solid support maybe column, a bead, or any other solid support known to those of skill in the art.

Other examples include the use of various kinds of chromatography. There are many kinds of chromatography that may be used in the practice of the present invention, including capillary adsorption, partition, ion-exchange, molecular sieve, reverse-phase, column, paper, thin-layer, and gas chromatography as well as HPLC. In particular, the analyte of interest may be removed by adsorption of the analyte onto a solid surface, such as silica or molecular sieves

Chromatography is used to separate organic compounds on the basis of their charge, size, shape, and solubilities. A chromatography consists of a mobile phase (solvent and the molecules to be separated) and a stationary phase either of paper (in paper chromatography) or glass beads, called resin, (in column chromatography) through which the mobile phase travels. Molecules travel through the stationary phase at different rates because of their chemistry. Types of chromatography that may be employed in the present invention include, but are not limited to, high performance liquid chromatography (HPLC), ion exchange chromatography (IEC), and reverse phase chromatography (RP). Other kinds of chromatography include: adsorption, partition, affinity, gel filtration and molecular sieve, and many specialized techniques for using them including column, paper, thin-layer and gas chromatography (Freifelder, 1982).

a. High Performance Liquid Chromatography

High performance liquid chromatography (HPLC) is similar to reverse phase, only in this method, the process is conducted at a high velocity and pressure drop. The column is shorter and has a small diameter, but it is equivalent to possessing a large number of equilibrium stages.

Although there are other types of chromatography (e.g., paper and thin layer), most applications of chromatography employ a column. The column is where the actual separation takes place. It is usually a glass or metal tube of sufficient strength to withstand the pressures that may be applied across it. The column contains the stationary phase. The mobile phase runs through the column and is adsorbed onto the stationary phase. The column can either be a packed bed or open tubular column. A packed bed column is comprised of a stationary phase which is in granular form and packed into the column as a homogeneous bed. The stationary phase completely fills the column. An open tubular column's stationary phase is a thin film or layer on the column wall. There is a passageway through the center of the column.

The mobile phase is comprised of a solvent into which the sample is injected. The solvent and sample flow through the column together; thus the mobile phase is often referred to as the “carrier fluid.” The stationary phase is the material in the column for which the components to be separated have varying affinities. The materials which comprise the mobile and stationary phases vary depending on the general type of chromatographic process being performed. The mobile phase in liquid chromatography is a liquid of low viscosity which flows through the stationary phase bed. This bed may be comprised of an immiscible liquid coated onto a porous support, a thin film of liquid phase bonded to the surface of a sorbent, or a sorbent of controlled pore size.

High-performance chromatofocusing (HPCF) produces liquid pI fractions as the first-dimension of protein separation followed by high-resolution reversed-phase (RP) HPLC of each of the pI fractions as the second dimension. Proteins are now mapped (like gels), but the liquid fractions make for easy interface with mass spectrometry (MS) for detailed intact protein characterization and identification (unlike gels) on more selective basis without resorting to protein digestion.

b. Reversed-Phase Chromatography

Reversed phase chromatography (RPC) utilizes solubility properties of the sample by partitioning it between a hydrophilic and a lipophilic solvent. The partition of the sample components between the two phases depends on their respective solubility characteristics. Less hydrophobic components end up primarily in the hydrophilic phase while more hydrophobic ones are found in the lipophilic phase. In RPC, silica particles covered with chemically-bonded hydrocarbon chains (2-18 carbons) represent the lipophilic phase, while an aqueous mixture of an organic solvent surrounding the particle represents the hydrophilic phase.

When a sample component passes through an RPC column the partitioning mechanism operates continuously. Depending on the extractive power of the eluent, a greater or lesser part of the sample component will be retained reversibly by the lipid layer of the particles, in this case called the stationary phase. The larger the fraction retained in the lipid layer, the slower the sample component will move down the column. Hydrophilic compounds will move faster than hydrophobic ones, since the mobile phase is more hydrophilic than the stationary phase.

Compounds stick to reverse phase HPLC columns in high aqueous mobile phase and are eluted from RP HPLC columns with high organic mobile phase. In RP HPLC compounds are separated based on their hydrophobic character. Peptides can be separated by running a linear gradient of the organic solvent.

Along with the partitioning mechanism, adsorption operates at the interface between the mobile and the stationary phases. The adsorption mechanism is more pronounced for hydrophilic sample components while for hydrophobic ones the liquid-liquid partitioning mechanism is prevailing. Thus the retention of hydrophobic components is greatly influenced by the thickness of the lipid layer. An 18 carbon layer is able to accommodate more hydrophobic material than an 8 carbon or a 2 carbon layer.

The mobile phase can be considered as an aqueous solution of an organic solvent, the type and concentration of which determines the extractive power. Some commonly used organic solvents, in order of increasing hydrophobicity are: methanol, propanol, acetonitrile, and tetrahydrofuran.

Due to the very small sizes of the particles employed as the stationary phase, very narrow peaks are obtained. In some embodiments, reverse phase HPLC peaks are represented by bands of different intensity in the two-dimensional image, according to the intensity of the peaks eluting from the HPLC. In some instances, peaks are collected as the eluent of the HPLC separation in the liquid phase. To improve the chromatographic peak shape and to provide a source of protons in reverse phase chromatography acids are commonly used. Such acids are formic acid, trifluoroacetic acid, and acetic acid.

c. Ion Exchange Chromatography

Ion exchange chromatography (IEC) is applicable to the separation of almost any type of charged molecule, from large proteins to small nucleotides and amino acids. It is very frequently used for proteins and peptides, under widely varying conditions. In protein structural work the consecutive use of gel permeation chromatography (GPC) and IEC is quite common.

In ion exchange chromatography, a charged particle (matrix) binds reversibly to sample molecules (proteins, etc.). Desorption is then brought about by increasing the salt concentration or by altering the pH of the mobile phase. Ion exchange containing diethyl aminoethyl (DEAE) or carboxymethyl (CM) groups are most frequently used in biochemistry. The ionic properties of both DEAE and CM are dependent on pH, but both are sufficiently charged to work well as ion exchangers within the pH range 4 to 8 where most protein separations take place.

The property of a protein which govern its adsorption to an ion exchanger is the net surface charge. Since surface charge is the result of weak acidic and basic groups of protein; separation is highly pH dependent. Going from low to high pH values the surface charge of proteins shifts from a positive to a negative charge surface charge. The pH versus net surface curve is a individual property of a protein, and constitutes the basis for selectivity in IEC.

As in all forms of liquid chromatography, conditions are employed that permit the sample components to move through the column with different speeds. At low ionic strengths, all components with affinity for the ion exchanger will be tightly adsorbed at the top of the ion exchanger and nothing will remain in the mobile phase. When the ionic strength of the mobile phase is increased by adding a neutral salt, the salt ions will compete with the protein and more of the sample components will be partially desorbed and start moving down the column. Increasing the ionic strength even more causes a larger number of the sample components to be desorbed, and the speed of the movement down the column will increase. The higher the net charge of the protein, the higher the ionic strength needed to bring about desorption. At a certain high level of ionic strength, all the sample components are fully desorbed and move down the column with the same speed as the mobile phase. Somewhere in between total adsorption and total desorption one will find the optimal selectivity for a given pH value of the mobile phase. Thus, to optimize selectivity in ion exchange chromatography, a pH value is chosen that creates sufficiently large net charge differences among the sample components. Then, an ionic strength is selected that fully utilizes these charge differences by partially desorbing the components. The respective speed of each component down the column will be proportional to that fraction of the component which is found in the mobile phase.

Very often the sample components vary so much in their adsorption to the ion exchanger that a single value of the ionic strength cannot make the slow ones pass through the column in a reasonable time. In such cases, a salt gradient is applied to bring about a continuous increase of ionic strength in the mobile phase.

3. Destroying the Analyte of Interest

In another embodiment, the analyte is removed from a sample or standard by destroying the analyte of interest. The analyte of interest may be destroyed by any method known to those of skill in the art to degrade the analyte. Such methods include but are not limited to treating the sample with a destructive agent or technique, such as a protease treatment or heat treatment.

4. Sequestering the Analyte of Interest

In still further embodiments, the analyte is removed from a sample or standard by sequestering the analyte of interest. The analyte of interest may be sequestered by any method known to those of skill in the art, including but not limited to incorporation of the analyte into a vehicle, such as a liposome.

In certain broad embodiments of the invention, the analyte of interest may be sequestered by entrapping the analyte in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are cationic lipid-nucleic acid complexes, such as lipofectamine-nucleic acid complexes.

“Liposome” is a generic term encompassing a variety of single and multilamellar lipid vehicles formed by the generation of enclosed lipid bilayers. Phospholipids are used for preparing the liposomes according to the present invention and can carry a net positive charge, a net negative charge or are neutral. Dicetyl phosphate can be employed to confer a negative charge on the liposomes, and stearylamine can be used to confer a positive charge on the liposomes.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem (La Jolla, Calif.); dimyristyl phosphatidylglycerol (“DMPG”) and other lipids may be obtained from Avanti Polar Lipids, Inc. (Birmingham, Ala.). Stock solutions of lipids in chloroform, chloroform/methanol or t-butanol can be stored at about −20.degree. C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

Liposomes used according to the present invention can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules will form a bilayer, known as a lamella, of the arrangement XY-YX.

Liposomes within the scope of the present invention can be prepared in accordance with known laboratory techniques. In one preferred embodiment, liposomes are prepared by mixing liposomal lipids, in a solvent in a container, e.g., a glass, pear-shaped flask. The container should have a volume ten-times greater than the volume of the expected suspension of liposomes. Using a rotary evaporator, the solvent is removed at approximately 40.degree. C. under negative pressure. The solvent normally is removed within about 5 min to 2 h, depending on the desired volume of the liposomes. The composition can be dried further in a desiccator under vacuum. The dried lipids generally are discarded after about 1 wk because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In the alternative, liposomes can be prepared in accordance with other known laboratory procedures: the method of Bangham et al. (1965), the contents of which are incorporated herein by reference; the method of Gregoriadis, as described in Drug Carriers in Biology and Medicine (1979), the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

The dried lipids or lyophilized liposomes prepared as described above may be reconstituted in a solution of nucleic acid and diluted to an appropriate concentration with an suitable solvent, e.g., DPBS. The mixture is then vigorously shaken in a vortex mixer. Unencapsulated nucleic acid is removed by centrifugation at 29,000.times.g and the liposomal pellets washed. The washed liposomes are resuspended at an appropriate total phospholipid concentration, e.g., about 50-200 mM. The amount of nucleic acid encapsulated can be determined in accordance with standard methods. After determination of the amount of nucleic acid encapsulated in the liposome preparation, the liposomes may be diluted to appropriate concentration and stored at 4.degree. C. until use.

### III. Multiplex Assays

Once created, the specifically deficient samples can be mixed in suitable proportions to produce a multiplex standard solution containing a predetermined concentration of each analyte of interest. In some embodiments, the invention further comprises making a series of dilutions of the multiplex standard. This series of dilutions may be used to create a standard curve for multiplex analysis of unknown samples. A standard curve may be generated by plotting assay data such as empirically determined values representative of a known concentration of an analyte. By relating similarly derived empirical values for analytes of unknown concentration in a sample to a standard curve, the concentration of analyte(s) in the sample, particularly proteins and DNA, may be calculated. The assay data may be obtained by any method known to those of skill in the art, including but not limited to those mentioned below.

A. Arrays

The present invention may involve the use of arrays to generate assay data for use in the invention. Array technology allows high-throughput screening for gene expression and molecular interactions. Protein array technology is discussed in detail in Pandey and Mann (2000) and MacBeath and Schreiber (2000), each of which is herein specifically incorporated by reference. These arrays, which typically contain thousands of different proteins or antibodies spotted onto glass slides or immobilized in tiny wells, allow one to examine the biochemical activities and binding profiles of a large number of proteins at once. To examine protein interactions with such an array, a labeled protein is incubated with each of the target proteins immobilized on the array. The array is then analyzed to determine which of the many proteins the labeled molecule binds, the quantity or concentration of the protein, or other characteristics of the protein. Those of skill in the art are aware of various methods available to analyze the array.

1. Protein Biochip Assays

Biochips, in general, comprise a substrate to which array of capture molecules has been attached, each at a discrete and identifiable location on the substrate surface in such a manner as to be addressable by a detection method of choice. When the capture molecules are exposed to an analytic sample, analytes in the sample can bind to a capture molecule on the surface for which it has affinity. The capture or interaction between an analyte molecule and a capture molecule is detected or characterized by any of a variety of means. Such detection or characterization methods are known to those of skill in the art, and include but are not limited to detection of fluorescence, luminescence, absorbance, reflectance, transmittance, or refractive index (e.g., surface plasmon resonance, ellipsometry, a resonant mirror method, a diffraction grating coupler waveguide method or interferometry), immunoassays (e.g., ELISA), gas phase ion spectrometry methods, atomic force microscopy or mass spectrometry and, in particular, SELDI. Quantification of the analytes in the sample can be achieved by selecting an appropriate method of detection.

2. Bead Arrays

Microsphere based assays may also be analyzed on bead array platforms. In general, bead array platforms image beads and analytes distributed on an array. In this way, imaging of bead arrays is similar to the gene chips discussed above. However, in contrast to gene chips where the analyte is identified by its spatial position on the array, bead arrays typically identify the analyte by the encoded microsphere to which it is bound.

For example, Luminex (Austin, Tex.) describe a method for encoding microspheres according to their fluorescence as taught in Fulton et al, 1997, Clin. Chem. 43:1749-1756 and U.S. Pat. No. 5,736,330 both of which are incorporated herein by reference. The methodology is based on the principle that fluorescent microspheres (beads) with unique fluorescent profiles can be immobilized to different analyte specific binders and used to create a fluorescence-based array of analyte specific beads where each bead type is specific for a unique analyte. This technology employs a combination of fluorescent dyes that allow each bead to be independently identified. The analyte specific microspheres are mixed together and contacted with a probe(s) that is labeled with a different fluorescent color. The probes bind to their ligands or receptors on the labeled microspheres and are used to determine the specific molecular interaction at the surface of each bead. The samples are read in a flow cytometer which allows each microsphere to be identified individually and the corresponding probe binding signal to be read.

The microspheres are available in 64 distinct sets that are classified by virtue of the unique orange/red emission profile of each set. Different concentrations of each of two fluorochromes, orange-emitting and red-emitting, were used to prepare 64 microsphere sets with unique orange/red emission profiles. The microspheres can be covalently coupled to virtually any amine-containing molecule through surface carboxylate groups. Alternatively, avidin-coupled microspheres are available for immobilizing biotinylated molecules (Fulton et al, 1997, Clin. Chem. 43: 1749-1756).

Other examples of commercially available bead arrays include Illumina's BeadXpress™ Reader and BeadStation 500™.

3. Antibody Microarrays

An antibody microarray is a specific form of protein microarrays. Antibody microarrays are often used in general research to detect protein expressions from cell lysates and may be used for diagnostic applications, for example for detecting special biomarkers from serum or urine.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### One Starting Sample

The following example demonstrates the creation of a multiplex standard for an assay having 14 analytes. A multiplex standard for the evaluation of 14 analytes is created from a single sample, S0. The level of each analyte in S0 is given in Table 1.

TABLE 1

| Analyte | Value (μg/ml) |
|---|---|
| PS-57 | 71,351 |
| PS-51 | 50,210 |
| PS-08 | 27,850 |
| PS-14 | 23,323 |
| PS-56 | 17,904 |
| PS-12 | 13,607 |
| PS-19 | 10,962 |
| PS-01 | 9,402 |
| PS-26 | 8,134 |
| PS-09 | 7,325 |
| PS-68 | 6,737 |
| PS-03 | 5,315 |
| PS-23 | 4,095 |
| PS-04 | 2,793 |

To create the multiplex standard, a set of standards is first created by systematically removing the analytes of interest from the sample. An analyte of interest may be removed by, for example, being bound to a solid support, thereby rendering a biological sample devoid or at least of low concentration of that particular analyte. This is generally done without affecting the analyte levels of the other analytes, however, this process does not need to be 100% specific nor 100% effective at removing the particular analyte as a residual amount may be acceptable. The set of standards may be created by sequentially removing the 14 analytes from the sample. For example, sample S0 may be treated to remove one analyte, PS-57. This creates a first specifically deficient sample, S1. S1 is then treated to remove a second analyte, PS-51 and create a second specifically deficient sample, S2. S2 is then treated to remove a third analyte, PS-08 and create a third specifically deficient sample, S3. S3 is then treated to remove a fourth analyte, PS-14 and create a fourth specifically deficient sample, S4. S4 is then treated to remove a fifth analyte, PS-56 and create a fifth specifically deficient sample, S5. S5 is then treated to remove a sixth analyte, PS-12 and create a sixth specifically deficient sample, S6. S6 is then treated to remove a seventh analyte, PS-19 and create a seventh specifically deficient sample, S7. S7 is then treated to remove an eighth analyte, PS-01 and create an eighth specifically deficient sample, S8. S8 is then treated to remove a ninth analyte, PS-26 and create a ninth specifically deficient sample, S9. S9 is then treated to remove a tenth analyte, PS-09 and create a tenth specifically deficient sample, S10, S10 is then treated to remove an eleventh analyte, PS-68 and create an eleventh specifically deficient sample, S11. S11 is then treated to remove a twelfth analyte, PS-03 and create a twelfth specifically deficient sample, S12. S12 is then treated to remove a thirteenth analyte, PS-23 and create a thirteenth specifically deficient sample, S13. Thus, there are 14 standards after this process, S0-S13, having the levels of analyte (μg/ml) found in Table 2.

TABLE 2

| | S0 | S1 | S2 | S3 | S4 | S5 | S6 | S7 | S8 | S9 | S10 | S11 | S12 | S13 |
|---|---|---|---|---|---|---|---|---|---|---|---|---|---|---|
| PS-57 | 71,351 | 0 | 0 | 0 | 0 | 0 | 0 | 0 | 0 | 0 | 0 | 0 | 0 | 0 |
| PS-51 | 50,210 | 50,210 | 0 | 0 | 0 | 0 | 0 | 0 | 0 | 0 | 0 | 0 | 0 | 0 |
| PS-08 | 27,850 | 27,850 | 27,850 | 0 | 0 | 0 | 0 | 0 | 0 | 0 | 0 | 0 | 0 | 0 |
| PS-14 | 23,323 | 23,323 | 23,323 | 23,323 | 0 | 0 | 0 | 0 | 0 | 0 | 0 | 0 | 0 | 0 |
| PS-56 | 17,904 | 17,904 | 17,904 | 17,904 | 17,904 | 0 | 0 | 0 | 0 | 0 | 0 | 0 | 0 | 0 |
| PS-12 | 13,607 | 13,607 | 13,607 | 13,607 | 13,607 | 13,607 | 0 | 0 | 0 | 0 | 0 | 0 | 0 | 0 |
| PS-19 | 10,962 | 10,962 | 10,962 | 10,962 | 10,962 | 10,962 | 10,962 | 0 | 0 | 0 | 0 | 0 | 0 | 0 |
| PS-01 | 9,402 | 9,402 | 9,402 | 9,402 | 9,402 | 9,402 | 9,402 | 9,402 | 0 | 0 | 0 | 0 | 0 | 0 |
| PS-26 | 8,134 | 8,134 | 8,134 | 8,134 | 8,134 | 8,134 | 8,134 | 8,134 | 8,134 | 0 | 0 | 0 | 0 | 0 |
| PS-09 | 7,325 | 7,325 | 7,325 | 7,325 | 7,325 | 7,325 | 7,325 | 7,325 | 7,325 | 7,325 | 0 | 0 | 0 | 0 |
| PS-68 | 6,737 | 6,737 | 6,737 | 6,737 | 6,737 | 6,737 | 6,737 | 6,737 | 6,737 | 6,737 | 6,737 | 0 | 0 | 0 |
| PS-03 | 5,315 | 5,315 | 5,315 | 5,315 | 5,315 | 5,315 | 5,315 | 5,315 | 5,315 | 5,315 | 5,315 | 5,315 | 0 | 0 |
| PS-23 | 4,095 | 4,095 | 4,095 | 4,095 | 4,095 | 4,095 | 4,095 | 4,095 | 4,095 | 4,095 | 4,095 | 4,095 | 4,095 | 0 |
| PS-04 | 2,793 | 2,793 | 2,793 | 2,793 | 2,793 | 2,793 | 2,793 | 2,793 | 2,793 | 2,793 | 2,793 | 2,793 | 2,793 | 2,793 |

Second, specific and calculated mixing of the standards, each now deficient in certain analytes, is performed. In particular, the amount of each standard required to create a multiplex standard is calculated based on the level of analyte in each standard. When the amounts calculated for each standard are mixed, a substantially uniform multiplex standard will be generated. Thus, to create a 100 ml multiplex standard, 7.008 ml of S0 plus 2.951 ml of S1 plus 7.995 ml of S2, and so forth as shown in Table 3, is mixed to a total of 100 ml.

TABLE 3

| Standard | ml required for 100 ml multiplex standard |
|---|---|
| S0 | 7.008 |
| S1 | 2.951 |
| S2 | 7.995 |
| S3 | 3.484 |
| S4 | 6.489 |
| S5 | 8.82 |
| S6 | 8.867 |
| S7 | 7.567 |
| S8 | 8.294 |
| S9 | 6.785 |
| S10 | 5.958 |
| S11 | 19.85 |
| S12 | 5.941 |
| S13 | 0.01 |

The concentration of each analyte in the 100 ml multiplex standard shown in Table 4 will be at the target value of 5,000 μg/ml with the exception of two (PS-23 and PS-04). Due to the nature of the biological material, target values can never exceed that of a given analyte as it is found in the highest level of any of the input samples. Thus, PS-23 and PS-04 cannot be at the desired concentration of 5,000 μg/ml level, as S0 does not have a value of PS-23 or PS-04 at or above 5,000 μg/ml.

TABLE 4

| Analyte | Value (μg/ml) |
|---|---|
| PS-01 | 5,000 |
| PS-03 | 5,000 |
| PS-04 | 2,793 |
| PS-08 | 5,000 |
| PS-09 | 5,000 |
| PS-12 | 5,000 |
| PS-14 | 5,000 |
| PS-19 | 5,000 |
| PS-23 | 4,095 |
| PS-26 | 5,000 |
| PS-51 | 5,000 |
| PS-56 | 5,000 |
| PS-57 | 5,000 |
| PS-68 | 5,000 |

To create a multiplex standard in which each analyte is at the target value, the target value should be set no higher than that of the analyte of lowest concentration. Under those circumstances, the target value of the multiplex standard could be achieved using a different mixing strategy based on the S0-S13 pools. To make 100 ml, an amount of each standard (S0-S13) as found in Table 5 is mixed to produce a multiplex standard in which all 14 analytes are at a concentration of approximately 2,793 μg/ml.

TABLE 5

| Standard | ml required for 100 ml standard |
|---|---|
| S0 | 3.914 |
| S1 | 1.648 |
| S2 | 4.466 |
| S3 | 1.946 |
| S4 | 3.625 |
| S5 | 4.927 |
| S6 | 4.953 |
| S7 | 4.227 |
| S8 | 4.633 |
| S9 | 3.79 |
| S10 | 3.328 |
| S11 | 11.088 |
| S12 | 15.665 |
| S13 | 31.807 |

The multiplex standard would then be systematically diluted to create a pool of multiplex standards.

Not all assays will require the creation of the full set of standards with reduced sample content. It may only be necessary to create a “partial” set of standards to reach the desired levels. This will be dependent on the actual starting material, as well as the target values desired. It is also not necessary to reduce the analyte level to zero; a residual value can be entirely acceptable in some situations.

### Example 2

### Five Starting Samples

A multiplex standard for identifying antibodies representative of 14 different serotype-specific antibodies against Pneumococcal serotypes (PS) in a serum sample is created from five starting samples, P1-P5.

The level of each of the 14 serotype-specific antibodies in the set of samples P1-P5 is given in Table 6. The five starting samples are blended together to create a multiplex standard. This results in concentrations of serotype-specific antibodies having the values shown in Table 7. Each serotype-specific antibody is present at a different concentration, and thus the multiplex standard is not uniform in terms of antibody concentration. If the target value is 5,000 μg/ml, the ratio of the actual concentration value of the serotype-specific antibody to the target concentration value of the serotype-specific antibody (“actual to target value ratio”) ranges from 0.30 to 1.90, with an average actual to target value ratio of 0.74. A ratio of 1 reflects a multiplex standard with an average concentration of 5,000 μg/ml of each serotype-specific antibody.

TABLE 6

| | P1 | P2 | P3 | P4 | P5 | | Total |
|---|---|---|---|---|---|---|---|
| Analyte | (μg/ml) | (μg/ml) | (μg/ml) | (μg/ml) | (μg/ml) | Diluent | Volume |
| Serotype 1 | 41,000 | 55,000 | 32,000 | 28,000 | 1,000 | 0 | |
| Antibody | | | | | | | |
| Serotype 2 | 900 | 36,500 | 11,300 | 6,800 | 66,100 | 0 | |
| Antibody | | | | | | | |
| Serotype 3 | 2,300 | 28,000 | 2,500 | 6,100 | 32,000 | 0 | |
| Antibody | | | | | | | |
| Serotype 4 | 6,200 | 23,200 | 11,500 | 7,400 | 18,400 | 0 | |
| Antibody | | | | | | | |
| Serotype 5 | 1,800 | 17,000 | 9,500 | 18,100 | 7,400 | 0 | |
| Antibody | | | | | | | |
| Serotype 6 | 2,300 | 13,000 | 7,000 | 10,600 | 3,700 | 0 | |
| Antibody | | | | | | | |
| Serotype 7 | 9,700 | 11,000 | 5,000 | 9,900 | 10,500 | 0 | |
| Antibody | | | | | | | |
| Serotype 8 | 14,600 | 9,000 | 3,000 | 4,900 | 27,800 | 0 | |
| Antibody | | | | | | | |
| Serotype 9 | 2,300 | 8,000 | 4,000 | 3,300 | 16,000 | 0 | |
| Antibody | | | | | | | |
| Serotype 10 | 8,000 | 6,000 | 11,000 | 10,000 | 23,000 | 0 | |
| Antibody | | | | | | | |
| Serotype 11 | 700 | 7,000 | 3,000 | 1,500 | 17,000 | 0 | |
| Antibody | | | | | | | |
| Serotype 12 | 2,000 | 5,000 | 1,000 | 2,800 | 14,000 | 0 | |
| Antibody | | | | | | | |
| Serotype 13 | 3,000 | 4,000 | 100 | 1,400 | 15,000 | 0 | |
| Antibody | | | | | | | |
| Serotype 14 | 2,900 | 2,793 | 12,000 | 9,100 | 49,000 | 0 | |
| Antibody | | | | | | | |
| ml of sample | 3 | 5 | 2 | 2 | 11 | 77 | 100 |

TABLE 7

| Analyte | Blend (μg/ml) | Ratio to target |
|---|---|---|
| Serotype 1-Ab | 5,290 | 1.06 |
| Serotype 2-Ab | 9,485 | 1.9 |
| Serotype 3-Ab | 5,161 | 1.03 |
| Serotype 4-Ab | 3,748 | 0.75 |
| Serotype 5-Ab | 2,270 | 0.45 |
| Serotype 6-Ab | 1,478 | 0.3 |
| Serotype 7-Ab | 2,294 | 0.46 |
| Serotype 8-Ab | 4,104 | 0.82 |
| Serotype 9-Ab | 2,375 | 0.48 |
| Serotype 10-Ab | 3,490 | 0.7 |
| Serotype 11-Ab | 2,331 | 0.47 |
| Serotype 12-Ab | 1,926 | 0.39 |
| Serotype 13-Ab | 1,970 | 0.39 |
| Serotype 14-Ab | 6,039 | 1.21 |

Ab—Antibodies

In contrast, removal of select analytes allows the creation of a multiplex standard in which the concentration of target analytes is more uniform, which is reflected in a lower actual to target value ratio than obtainable by mixing the five starting samples. In particular, the set of standards is created by removing select analytes from the starting samples, P1-P5. For example, P1 is treated to remove serotype 1-specific antibodies, P2 is treated to remove serotype 3-specific antibodies, P3 is treated to remove serotype 14-specific antibodies, P4 is treated to remove serotype 5-specific antibodies, and P5 is treated to remove serotype 2-specific antibodies. P1-P5 will then have the levels (μg/ml) of target analytes (serotype-specific antibodies) found in Table 8.

TABLE 8

| | P1 | P2 | P3 | P4 | P5 | | Total |
|---|---|---|---|---|---|---|---|
| Analyte | (μg/ml) | (μg/ml) | (μg/ml) | (μg/ml) | (μg/ml) | Diluent | Volume |
| Serotype 1 | 0 | 55,000 | 32,000 | 28,000 | 1,000 | 0 | |
| Antibodies | | | | | | | |
| Serotype 2 | 900 | 36,500 | 11,300 | 6,800 | 0 | 0 | |
| Antibodies | | | | | | | |
| Serotype 3 | 2,300 | 0 | 2,500 | 6,100 | 32,000 | 0 | |
| Antibodies | | | | | | | |
| Serotype 4 | 6,200 | 23,200 | 11,500 | 7,400 | 18,400 | 0 | |
| Antibodies | | | | | | | |
| Serotype 5 | 1,800 | 17,000 | 9,500 | 0 | 7,400 | 0 | |
| Antibodies | | | | | | | |
| Serotype 6 | 2,300 | 13,000 | 7,000 | 10,600 | 3,700 | 0 | |
| Antibodies | | | | | | | |
| Serotype 7 | 9,700 | 11,000 | 5,000 | 9,900 | 10,500 | 0 | |
| Antibodies | | | | | | | |
| Serotype 8 | 14,600 | 9,000 | 3,000 | 4,900 | 27,800 | 0 | |
| Antibodies | | | | | | | |
| Serotype 9 | 2,300 | 8,000 | 4,000 | 3,300 | 16,000 | 0 | |
| Antibodies | | | | | | | |
| Serotype 10 | 8,000 | 6,000 | 11,000 | 10,000 | 23,000 | 0 | |
| Antibodies | | | | | | | |
| Serotype 11 | 700 | 7,000 | 3,000 | 1,500 | 17,000 | 0 | |
| Antibodies | | | | | | | |
| Serotype 12 | 2,000 | 5,000 | 1,000 | 2,800 | 14,000 | 0 | |
| Antibodies | | | | | | | |
| Serotype 13 | 3,000 | 4,000 | 100 | 1,400 | 15,000 | 0 | |
| Antibodies | | | | | | | |
| Serotype 14 | 2,900 | 2,793 | 0 | 9,100 | 49,000 | 0 | |
| Antibodies | | | | | | | |
| ml of sample | 12 | 6 | 2 | 2 | 16 | 62 | 100 |

The five specifically deficient samples are then blended together to create a multiplex standard. This results in a standard having serotype-specific antibodies with the concentration values demonstrated in Table 9. If the target value is 5,000 μg/ml, the actual to target value ratio ranges from 0.40 to 1.71, with an average actual to target value ratio of 0.86. The ideal actual value to target value ratio is 1.0. Thus, this approach makes it possible to achieve an average actual concentration value that is closer to a predetermined ideal concentration value than can be obtained using standard mixing of unmodified starting samples.

In this example, the removal of additional serotype-specific antibodies would result in a better actual versus target ratio and a smaller range of concentration values. Similarly, selecting several serotype-specific antibodies to be removed per sample would further enhance the actual versus target ratio and a smaller range, and thus create an even more uniform standard.

TABLE 9

| | Blend (μg/ml) | Ratio to target |
|---|---|---|
| Serotype 1 Antibodies | 4,660 | 0.93 |
| Serotype 2 Antibodies | 2,660 | 0.53 |
| Serotype 3 Antibodies | 5,568 | 1.11 |
| Serotype 4 Antibodies | 5,458 | 1.09 |
| Serotype 5 Antibodies | 2,610 | 0.52 |
| Serotype 6 Antibodies | 2,000 | 0.4 |
| Serotype 7 Antibodies | 3,802 | 0.76 |
| Serotype 8 Antibodies | 6,898 | 1.38 |
| Serotype 9 Antibodies | 3,462 | 0.69 |
| Serotype 10 Antibodies | 5,420 | 1.08 |
| Serotype 11 Antibodies | 3,314 | 0.66 |
| Serotype 12 Antibodies | 2,856 | 0.57 |
| Serotype 13 Antibodies | 3,030 | 0.61 |
| Serotype 14 Antibodies | 8,538 | 1.71 |

### Example 3

### Ten Starting Samples

A multiplex standard having 14 analytes present at a predetermined concentration is prepared from ten starting samples, P1-P10. As in Example 2, the multiplex standard may be useful in identification of multiple Pneumococcal serotypes, and the standard may evaluate the levels of 14 different serotype-specific antibodies against Pneumococcal serotypes in a serum sample.

The level of each of the 14 serotype-specific antibodies in a set of samples P1-P10 is provided in Table 10. The ten starting samples are blended together to create a multiplex standard. This results in serotype-specific antibodies present at the concentration values shown in Table 10. Each serotype-specific antibody is present at a different concentration, and thus the multiplex standard is not uniform in terms of the concentration of the 14 different serotype-specific antibodies. If the target concentration value is 5,000 μg/ml, the actual to target value ratio ranges from 0.59 to 1.73, with an average actual to target value ratio of 1.01.

TABLE 10

| | P1 | P2 | P3 | P4 | P5 | P6 | P7 | P8 | P9 | P10 | | Total |
|---|---|---|---|---|---|---|---|---|---|---|---|---|
| Analyte | (μg/ml) | (μg/ml) | (μg/ml) | (μg/ml) | (μg/ml) | (μg/ml) | (μg/ml) | (μg/ml) | (μg/ml) | (μg/ml) | Diluent | Volume |
| Serotype 1 | 41,000 | 55,000 | 32,000 | 28,000 | 1,000 | 15,000 | 16,000 | 20,000 | 21,700 | 9,900 | 0 | |
| Antibodies | | | | | | | | | | | | |
| Serotype 2 | 900 | 36,500 | 11,300 | 6,800 | 66,100 | 2,200 | 6,100 | 11,500 | 3,500 | 4,900 | 0 | |
| Antibodies | | | | | | | | | | | | |
| Serotype 3 | 2,300 | 28,000 | 2,500 | 6,100 | 32,000 | 12,900 | 900 | 9,500 | 7,300 | 3,300 | 0 | |
| Antibodies | | | | | | | | | | | | |
| Serotype 4 | 6,200 | 23,200 | 11,500 | 7,400 | 18,400 | 9,900 | 2,600 | 16,500 | 7,100 | 6,400 | 0 | |
| Antibodies | | | | | | | | | | | | |
| Serotype 5 | 1,800 | 17,000 | 9,500 | 18,100 | 7,400 | 23,000 | 9,200 | 6,500 | 1,600 | 1,500 | 0 | |
| Antibodies | | | | | | | | | | | | |
| Serotype 6 | 2,300 | 13,000 | 7,000 | 10,600 | 3,700 | 1,000 | 13,200 | 14,100 | 16,000 | 2,800 | 0 | |
| Antibodies | | | | | | | | | | | | |
| Serotype 7 | 9,700 | 11,000 | 5,000 | 9,900 | 10,500 | 10,000 | 7,900 | 6,800 | 7,400 | 1,400 | 0 | |
| Antibodies | | | | | | | | | | | | |
| Serotype 8 | 14,600 | 9,000 | 3,000 | 4,900 | 27,800 | 3,300 | 24,000 | 21,700 | 3,700 | 6,100 | 0 | |
| Antibodies | | | | | | | | | | | | |
| Serotype 9 | 2,300 | 8,000 | 4,000 | 3,300 | 16,000 | 2,100 | 1,400 | 3,500 | 10,500 | 1,600 | 0 | |
| Antibodies | | | | | | | | | | | | |
| Serotype 10 | 8,000 | 6,000 | 11,000 | 10,000 | 23,000 | 20,000 | 900 | 7,300 | 27,800 | 900 | 0 | |
| Antibodies | | | | | | | | | | | | |
| Serotype 11 | 700 | 7,000 | 3,000 | 1,500 | 17,000 | 2,500 | 11,600 | 7,100 | 16,000 | 2,600 | 0 | |
| Antibodies | | | | | | | | | | | | |
| Serotype 12 | 2,000 | 5,000 | 1,000 | 2,800 | 14,000 | 8,300 | 2,800 | 1,600 | 11,000 | 9,200 | 0 | |
| Antibodies | | | | | | | | | | | | |
| Serotype 13 | 3,000 | 4,000 | 100 | 1,400 | 15,000 | 4,200 | 1,500 | 16,000 | 9,000 | 13,200 | 0 | |
| Antibodies | | | | | | | | | | | | |
| Serotype 14 | 2,900 | 2,793 | 12,000 | 9,100 | 49,000 | 4,600 | 6,400 | 14,000 | 8,000 | 7,900 | 0 | |
| Antibodies | | | | | | | | | | | | |
| ml of pt sample | 2 | 1 | 3 | 3 | 10 | 8 | 3 | 3 | 6 | 4 | 57 | 100 |

TABLE 11

| Analyte | Blend (μg/ml) | Ratio to target |
|---|---|---|
| Serotype 1 Ab | 7,248 | 1.45 |
| Serotype 2 Ab | 8,646 | 1.73 |
| Serotype 3 Ab | 5,698 | 1.14 |
| Serotype 4 Ab | 4,810 | 0.96 |
| Serotype 5 Ab | 4,241 | 0.85 |
| Serotype 6 Ab | 3,045 | 0.61 |
| Serotype 7 Ab | 3,542 | 0.71 |
| Serotype 8 Ab | 5,500 | 1.1 |
| Serotype 9 Ab | 2,954 | 0.59 |
| Serotype 10 Ab | 6,700 | 1.34 |
| Serotype 11 Ab | 3,744 | 0.75 |
| Serotype 12 Ab | 3,428 | 0.69 |
| Serotype 13 Ab | 3,574 | 0.71 |
| Serotype 14 Ab | 7,395 | 1.48 |

Ab—Antibodies

In contrast, removal of select analytes (in this example serotype-specific antibodies) allows for the preparation of a standard having more uniform concentrations of the 14 different analytes, reflected in a lower actual to target value ratio than is obtainable by standard mixing of the ten unmodified starting samples. In particular, the set of standards is created by sequentially removing select analytes from the starting samples, P1-P10. For example, P2 is treated to remove serotype 1-specific antibodies, P4 is treated to remove serotype 5-specific antibodies, P5 is treated to remove serotype 2-specific antibodies, P6 is treated to remove serotype 3-specific antibodies, P8 is treated to remove serotype 4-specific antibodies, and P9 is treated to remove serotype 6-specific antibodies. P1-P10 will then have the levels of analyte (μg/ml) shown in Table 12.

TABLE 12

| | P1 | P2 | P3 | P4 | P5 | P6 | P7 | P8 | P9 | P10 | | Total |
|---|---|---|---|---|---|---|---|---|---|---|---|---|
| | (μg/ml) | (μg/ml) | (μg/ml) | (μg/ml) | (μg/ml) | (μg/ml) | (μg/ml) | (μg/ml) | (μg/ml) | (μg/ml) | Diluent | Volume |
| Serotype 1 Ab | 41,000 | 0 | 32,000 | 28,000 | 1,000 | 15,000 | 16,000 | 20,000 | 21,700 | 9,900 | 0 | |
| Serotype 2 Ab | 900 | 36,500 | 11,300 | 6,800 | 0 | 2,200 | 6,100 | 11,500 | 3,500 | 4,900 | 0 | |
| Serotype 3 Ab | 2,300 | 28,000 | 2,500 | 6,100 | 32,000 | 0 | 900 | 9,500 | 7,300 | 3,300 | 0 | |
| Serotype 4 Ab | 6,200 | 23,200 | 11,500 | 7,400 | 18,400 | 9,900 | 2,600 | 0 | 7,100 | 6,400 | 0 | |
| Serotype 5 Ab | 1,800 | 17,000 | 9,500 | 0 | 7,400 | 23,000 | 9,200 | 6,500 | 1,600 | 1,500 | 0 | |
| Serotype 6 Ab | 2,300 | 13,000 | 7,000 | 10,600 | 3,700 | 1,000 | 13,200 | 14,100 | 0 | 2,800 | 0 | |
| Serotype 7 Ab | 9,700 | 11,000 | 5,000 | 9,900 | 10,500 | 10,000 | 7,900 | 6,800 | 7,400 | 1,400 | 0 | |
| Serotype 8 Ab | 14,600 | 9,000 | 3,000 | 4,900 | 27,800 | 3,300 | 24,000 | 21,700 | 3,700 | 6,100 | 0 | |
| Serotype 9 Ab | 2,300 | 8,000 | 4,000 | 3,300 | 16,000 | 2,100 | 1,400 | 3,500 | 10,500 | 1,600 | 0 | |
| Serotype 10 Ab | 8,000 | 6,000 | 11,000 | 10,000 | 23,000 | 20,000 | 900 | 7,300 | 27,800 | 900 | 0 | |
| Serotype 11 Ab | 700 | 7,000 | 3,000 | 1,500 | 17,000 | 2,500 | 11,600 | 7,100 | 16,000 | 2,600 | 0 | |
| Serotype 12 Ab | 2,000 | 5,000 | 1,000 | 2,800 | 14,000 | 8,300 | 2,800 | 1,600 | 11,000 | 9,200 | 0 | |
| Serotype 13 Ab | 3,000 | 4,000 | 100 | 1,400 | 15,000 | 4,200 | 1,500 | 16,000 | 9,000 | 13,200 | 0 | |
| Serotype 14 Ab | 2,900 | 2,793 | 0 | 9,100 | 49,000 | 4,600 | 6,400 | 14,000 | 8,000 | 7,900 | 0 | |
| ml of pt sample | 0 | 8 | 2 | 1 | 7 | 5 | 8 | 5 | 7 | 5 | 52 | 100 |

Ab—Antibodies

The ten specifically deficient samples are then blended together to create a multiplex standard having predetermined concentrations of multiple analytes. This results in a standard having serotype-specific antibodies present at concentration values shown in Table 13. If the target value is 5,000 μg/ml, the actual to target value ratio ranges from 0.62 to 1.3, with an average actual to target value ratio of 0.95.

As with the standard created with five starting samples, removing additional serotype-specific antibodies or removing several serotype-specific antibodies per sample would result in a better actual versus target ratio and a smaller range, thus creating a more uniform standard.

TABLE 13

| | Blend (μg/ml) | Ratio to target |
|---|---|---|
| Serotype 1 Ab | 6,034 | 1.21 |
| Serotype 2 Ab | 4,877 | 0.98 |
| Serotype 3 Ab | 5,814 | 1.16 |
| Serotype 4 Ab | 4,968 | 0.99 |
| Serotype 5 Ab | 4,466 | 0.89 |
| Serotype 6 Ab | 3,496 | 0.7 |
| Serotype 7 Ab | 3,874 | 0.77 |
| Serotype 8 Ab | 6,509 | 1.3 |
| Serotype 9 Ab | 3,080 | 0.62 |
| Serotype 10 Ab | 5,838 | 1.17 |
| Serotype 11 Ab | 4,483 | 0.9 |
| Serotype 12 Ab | 3,377 | 0.68 |
| Serotype 13 Ab | 3,806 | 0.76 |
| Serotype 14 Ab | 6,141 | 1.23 |

Ab—Antibodies

### Example 4

### Removal of Pneumococcal Polysaccharide-Binding Antibodies to Create Specifically Deficient Samples

Two approaches were used to create samples specifically deficient in antibodies to a particular pneumococcal polysaccharide. In one approach, the removal of an antibody serotype was achieved by immobilizing the antibody on a column having a pneumococcal antigen specific for the pneumococcal antibodies present in the serotype. The columns were prepared by conjugating individual pneumococcal polysaccharides to crosslinked agarose beads using hydrozine/oxidized carbohydrate chemistry. The affinity chromatography media was then loaded into 1 ml spin columns. The resulting sample was tested for the ability of the column to remove the analyte of interest, and the effect of the affinity chromatography resin was compared with a control resin without the specific polysaccharide.

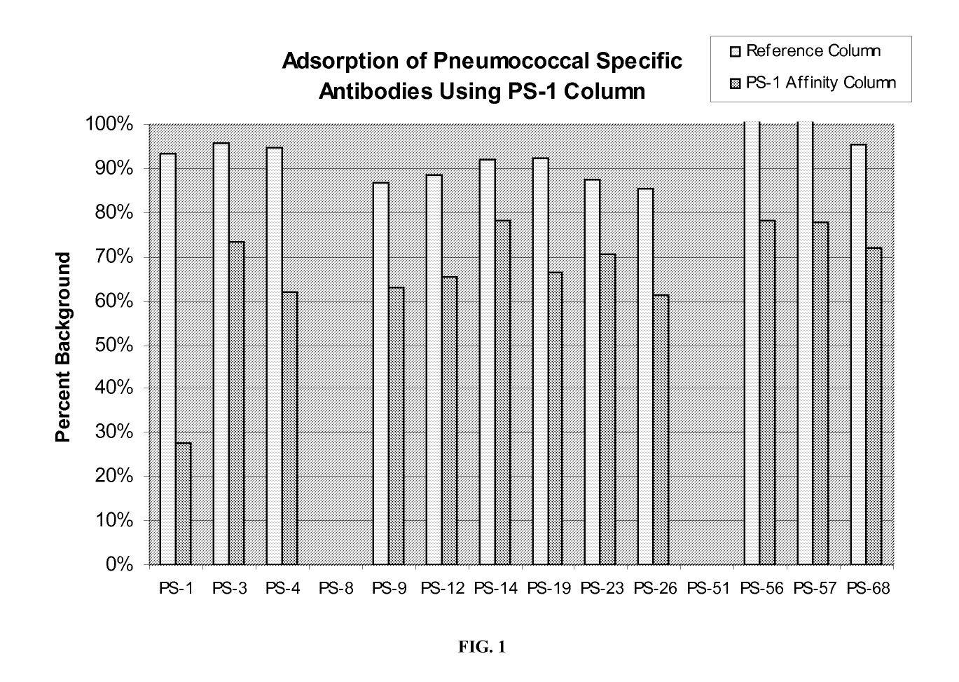

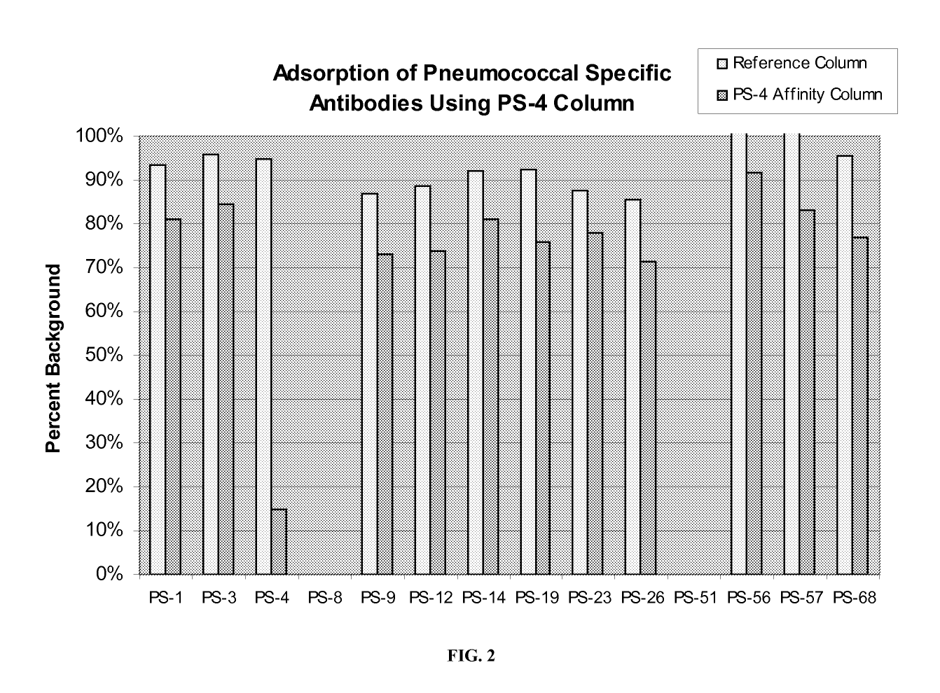

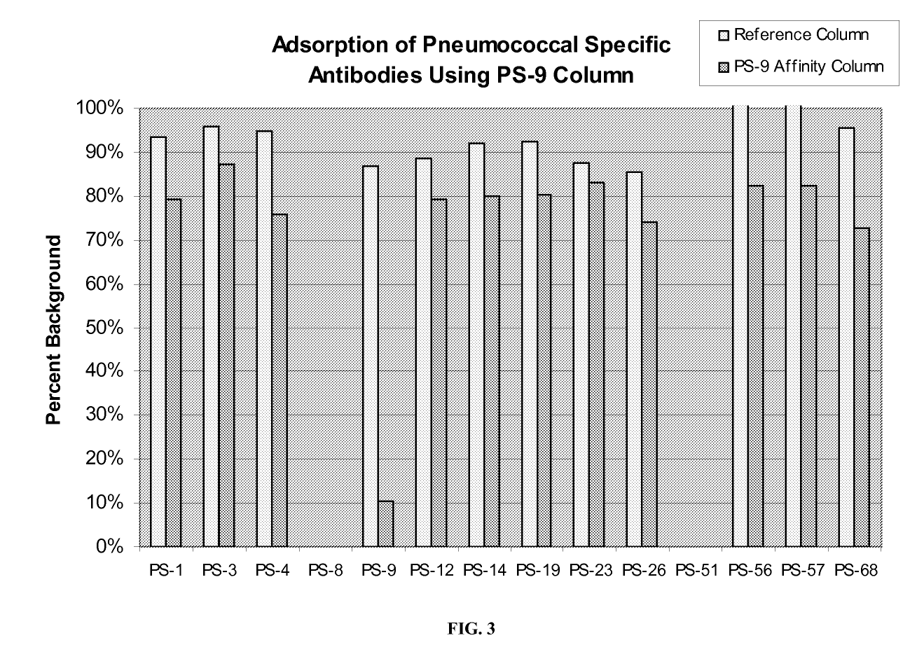

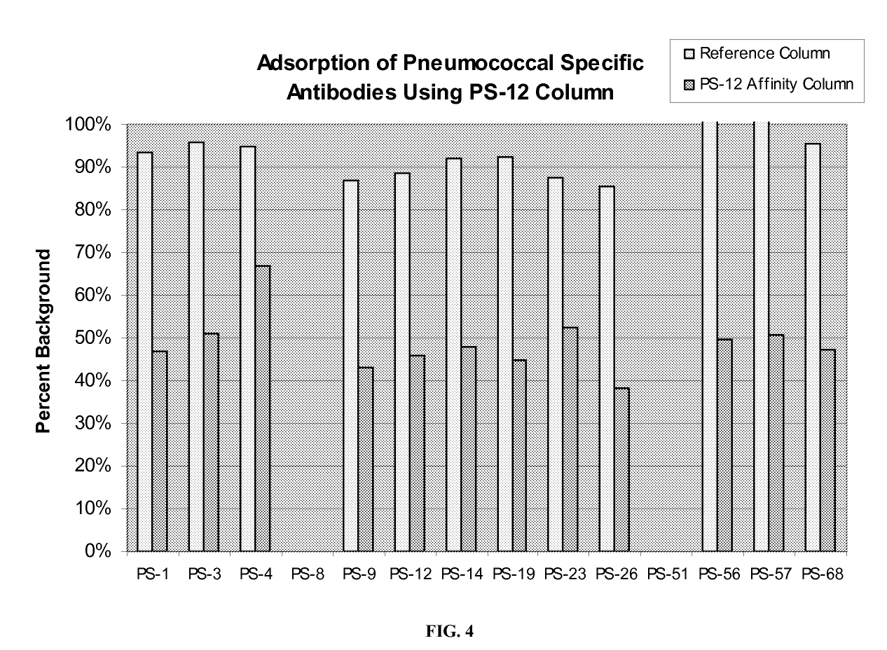

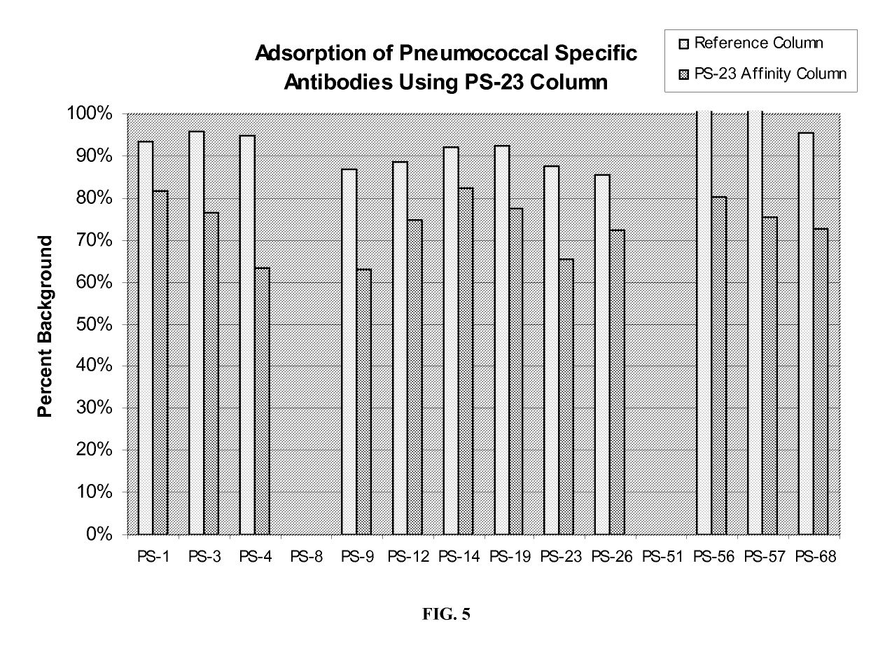

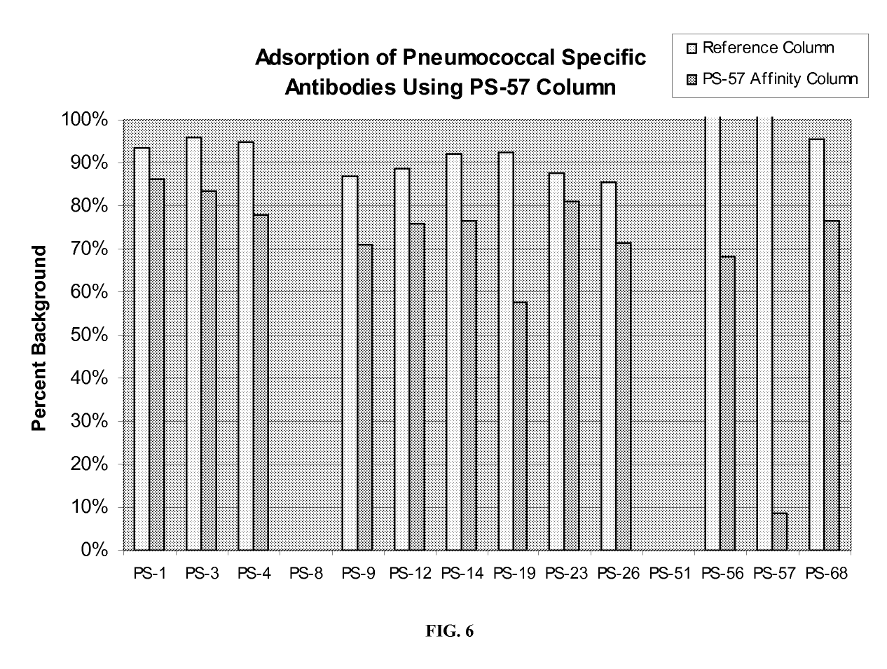

As shown in FIGS. 1-3 and 6, antibodies to PS-1, PS-4, PS-9, and PS-57 were specifically reduced to less than 30% of the antibody originally present in the sample using the specific affinity columns. As discussed above, it is not necessary to completely eliminate a specific analyte to create a specifically deficient sample for use in a multianalyte standard. Reduction of the PS-1, PS-4, PS-9, and PS-57 antibodies to less than 30% of the antibody originally present in the sample is a sufficient reduction in this example. The PS-12 affinity column non-specifically reduced antibodies to almost all of the antigens, as shown in FIG. 4. The PS-23 affinity column had a limited effect on all of the Pneumococcal antibodies (FIG. 5).

In another approach, the removal of specific pneumococcal serotypes was achieved by adding a binding agent to the sample to remove the analyte of interest, where the binding agent was a pneumococcal polysaccharide antigen specific for the pneumococcal antibodies present in the serotype of interest. The resulting sample was tested for the ability of the binding agent to remove the analyte of interest.

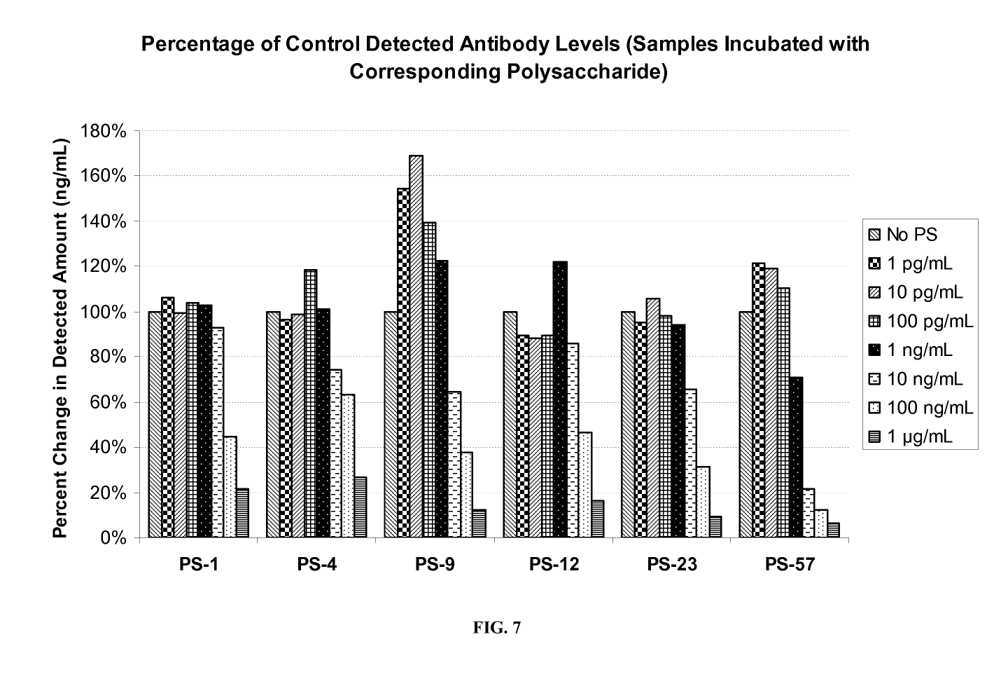

By adding the antigen to the sample, the analyte of interest was bound to the antigen, resulting in the analyte of interest being unavailable. As shown in Tables 14, 15, and 16 and FIG. 7, samples specifically deficient in an analyte of interest were successfully created for the PS-1, PS-4, PS-9, PS-12, PS-23, and PS-57 serotypes when 1 ug/ml of the corresponding polysaccharide was used to bind the antibodies. Lower amounts of polysaccharide also resulted in specifically deficient samples for certain analytes (Tables 14, 15, and 16 and FIG. 7)

TABLE 14

| | Amt PS | PS-1 | PS-4 | PS-9 | PS-12 | PS-23 | PS-57 |
|---|---|---|---|---|---|---|---|
| Amt PS | (g/mL) | (ng/mL) | (ng/mL) | (ng/mL) | (ng/mL) | (ng/mL) | (ng/mL) |
| 0 | 0.E+00 | 35.26 | 11.59 | 24.83 | 34.62 | 25.58 | 93.76 |
| 1 pg/mL | 1.E−12 | 37.48 | 11.17 | 38.32 | 31.03 | 24.29 | 113.83 |
| 10 pg/mL | 1.E−11 | 35.07 | 11.47 | 41.90 | 30.56 | 26.99 | 111.81 |
| 100 pg/mL | 1.E−10 | 36.59 | 13.75 | 34.57 | 31.04 | 25.14 | 103.71 |
| 1 ng/mL | 1.E−09 | 36.33 | 11.74 | 30.36 | 42.24 | 24.08 | 66.42 |
| 10 ng/mL | 1.E−08 | 32.71 | 8.61 | 16.01 | 29.77 | 16.80 | 20.00 |
| 100 ng/mL | 1.E−07 | 15.70 | 7.33 | 9.39 | 16.02 | 8.08 | 11.25 |
| 1 μg/mL | 1.E−06 | 7.50 | 3.08 | 3.08 | 5.66 | 2.33 | 5.81 |

TABLE 15

| [PS] | PS-1 | PS-4 | PS-9 | PS-12 | PS-23 | PS-57 |
|---|---|---|---|---|---|---|
| No PS | 0.00% | 0.00% | 0.00% | 0.00% | 0.00% | 0.00% |
| 1 pg/mL | 6.30% | −3.62% | 54.33% | −10.37% | −5.04% | 21.41% |
| 10 pg/mL | −0.54% | −1.04% | 68.75% | −11.73% | 5.51% | 19.25% |
| 100 pg/mL | 3.77% | 18.64% | 39.23% | −10.34% | −1.72% | 10.61% |
| 1 ng/mL | 3.03% | 1.29% | 22.27% | 22.01% | −5.86% | −29.16% |
| 10 ng/mL | −7.23% | −25.71% | −35.52% | −14.01% | −34.32% | −78.67% |
| 100 ng/mL | −55.47% | −36.76% | −62.18% | −53.73% | −68.41% | −88.00% |
| 1 μg/mL | −78.73% | −73.43% | −87.60% | −83.65% | −90.89% | −93.80% |

TABLE 16

| [PS] | PS-1 | PS-4 | PS-9 | PS-12 | PS-23 | PS-57 |
|---|---|---|---|---|---|---|
| No PS | 100.00% | 100.00% | 100.00% | 100.00% | 100.00% | 100.00% |
| 1 pg/mL | 106.30% | 96.38% | 154.33% | 89.63% | 94.96% | 121.41% |
| 10 pg/mL | 99.46% | 98.96% | 168.75% | 88.27% | 105.51% | 119.25% |
| 100 pg/mL | 103.77% | 118.64% | 139.23% | 89.66% | 98.28% | 110.61% |
| 1 ng/mL | 103.03% | 101.29% | 122.27% | 122.01% | 94.14% | 70.84% |
| 10 ng/mL | 92.77% | 74.29% | 64.48% | 85.99% | 65.68% | 21.33% |
| 100 ng/mL | 44.53% | 63.24% | 37.82% | 46.27% | 31.59% | 12.00% |
| 1 μg/mL | 21.27% | 26.57% | 12.40% | 16.35% | 9.11% | 6.20% |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
Bangham et al., J. Mol. Biol., 13(1):238-252; 253-259, 1965.
De Jager et al., Semin. Nucl. Med., 23(2):165-179, 1993.
Deamer and Uster, In: Liposome Preparation: Methods and Mechanisms, Ostro (Ed.), Liposomes, 1983.
Doolittle and Ben-Zeev, Methods Mol. Biol, 109:215-237, 1999.
Freifelder, In: Physical Biochemistry Applications to Biochemistry and Molecular Biology, 2nd Ed. Wm. Freeman and Co., NY, 1982.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gregoriadis, In: Drug Carriers in Biology and Medicine, Gregoriadis (Ed.), 287-341, 1979.
Gulbis and Galand, Hum. Pathol., 24(12):1271-1285, 1993.
MacBeath and Schreiber, Science, 289(5485):1760-1763, 2000.
Nakamura et al., In: Handbook of Experimental Immunology (4^{th} Ed.), Weir et al., (Eds). 1:27, Blackwell Scientific Publ., Oxford, 1987.
Pandey and Mann, Nature, 405(6788):837-846, 2000.
Szoka and Papahadjopoulos, Proc. Natl. Acad. Sci. USA, 75:4194-4198, 1978.

1. A method for creating a multiplex standard for a plurality of analytes from a starting sample comprising:
(a) obtaining a sample comprising a plurality of analytes of interest;
(b) determining a concentration for each analyte of interest;
(c) creating one or more specifically deficient samples;
(d) determining an amount of the starting sample and of each specifically deficient sample required to create a multiplex standard having a substantially uniform concentration of each analyte; and
(e) mixing the amount of the starting sample and the specifically deficient samples to create the multiplex standard.

2. The method of claim 1, wherein creating the one or more specifically deficient samples comprises treating a portion of the starting sample to remove at least 70% of a first analyte of interest.

3. The method of claim 1, wherein creating the one or more specifically deficient samples comprises:
(a) treating a portion of the starting sample to remove a first analyte of interest to create a first specifically deficient sample; and
(b) treating a portion of the first specifically deficient sample to remove a second analyte of interest to create a second specifically deficient sample.

4. The method of claim 3, wherein the first analyte of interest is the analyte having the highest concentration in the starting sample and the second analyte of interest is the analyte having the highest concentration in the first specifically deficient sample.

5. The method of claim 3, wherein creating the one or more specifically deficient samples further comprises:
(c) treating a portion of the second specifically deficient sample to remove a third analyte of interest to create a third specifically deficient sample; and
(d) treating a portion of the third specifically deficient sample to remove a fourth analyte of interest to create a fourth specifically deficient sample.

6. The method of claim 5, wherein the first analyte of interest is the analyte having the highest concentration in the starting sample, the second analyte of interest is the analyte having the highest concentration in the first specifically deficient sample, the third analyte of interest is the analyte with the highest concentration in the second specifically deficient sample, and the fourth analyte of interest is the analyte with the highest concentration in the third specifically deficient sample.

7. The method of claim 2, wherein the treatment comprises physically removing the analyte or neutralizing the analyte.

8. The method of claim 7, wherein physically removing the analyte comprises immobilizing the analyte on a solid support.

9. The method of claim 7, wherein neutralizing the analyte comprises providing the analyte with a target molecule that removes the reactivity of the analyte.

10. The method of claim 2, wherein the starting sample comprises at least 3 analytes of interest.

11. The method of claim 1, wherein the starting sample is a blood sample.

12. The method of claim 1, wherein at least one analyte of interest is an antibody.

13. The method of claim 1, wherein each analyte in the multiplex standard has a ratio of actual concentration to target concentration of between 0.5 and 1.5.

14. The method of claim 1, further comprising making a series of dilutions of the multiplex standard.

15. The method of claim 1, wherein creating the one or more specifically deficient samples comprises:
(i) identifying one or more analytes of interest that have outlier concentrations; and
(ii) treating a portion of the starting sample to remove at least 70% of the one or more analytes of interest that have outlier concentrations to create one or more specifically deficient samples.

16. A method for creating a multiplex standard for a plurality of analytes from a plurality of starting samples comprising:
(a) obtaining a plurality of starting samples comprising a plurality of analytes of interest;
(b) determining a concentration for each analyte of interest in each starting sample;
(c) creating one or more specifically deficient samples;
(d) determining the amount of each specifically deficient sample required to create a multiplex standard having a desired concentration of each analyte; and
(e) mixing the amount of the starting sample and the specifically deficient samples to create the multiplex standard.

17. The method of claim 16, wherein creating the one or more specifically deficient samples comprises treating a portion of at least one of the starting samples to remove at least 70% of at least one analyte of interest.

18. The method of claim 16, wherein creating the one or more specifically deficient samples comprises:
(a) treating a portion of a first starting samples to remove a first analyte of interest to create a first specifically deficient sample; and
(b) treating a portion of a second starting sample to remove a second analyte of interest to create a second specifically deficient sample.

19. The method of claim 18, wherein the first analyte of interest is the analyte having the highest concentration in the first starting sample and the second analyte of interest is the analyte having the highest concentration in the second starting sample.

20. The method of claim 16, wherein the multiplex standard is created from at least 5 starting samples.

21. The method of claim 16, wherein creating the one or more specifically deficient samples comprises:
(i) identifying one or more analytes of interest that have outlier concentrations; and
(ii) treating a portion of at least one of the starting samples to remove at least 70% of the one or more analytes of interest that have outlier concentrations to create one or more specifically deficient samples.
